(19) United States
(12) Patent Application Publication
(10) Pub. No.: US 2021/0113143 A1
(43) Pub. Date: Apr. 22, 2021

(54) MOVEMENT DISORDER DETERMINATION SYSTEMS AND METHODS

(71) Applicants: Ryan Richard Grunsten, Whiting, IN (US); Rasvik Kudum, Ashburn, VA (US); Charles Joseph Pisciotta, Boonton Township, NJ (US); Yug Nikhilkumar Rao, Glen Allen, VA (US)

(72) Inventors: Ryan Richard Grunsten, Whiting, IN (US); Rasvik Kudum, Ashburn, VA (US); Charles Joseph Pisciotta, Boonton Township, NJ (US); Yug Nikhilkumar Rao, Glen Allen, VA (US)

(21) Appl. No.: 16/656,501

(22) Filed: Oct. 17, 2019

(51) Int. Cl. A61B 5/00; G16H 50/20; G06N 3/08; A61B 5/11

(52) U.S. Cl. CPC A61B 5/4082; G16H 50/20; A61B 5/6898; A61B 5/1124; A61B 5/7264; G06N 3/08

(57) ABSTRACT

The present invention is directed to movement disorder determination systems and methods. The systems and methods involve a mobile device and a computer system communicating with each other over a communications network. The mobile device is implemented with a pattern receiving and conversion software application and the computer system is implemented with a pattern processing software application. The pattern receiving and conversion software application and the pattern processing software application are configured to determine whether an individual has a movement disorder and the severity of the disorder if he does from a trace produced by the individual over a pattern such as an Archemidal spiral. The pattern processing software application processes the user trace to make the determination and informs the user the result of its determination.

### FIELD OF THE INVENTION

The present invention relates to the field of diagnostics. In particular, the present invention concerns systems and methods for determining whether an individual has a movement disorder by analyzing a trace he produced on a pattern such as a Archimedean spiral.

### BACKGROUND OF THE INVENTION

Movement disorders such as Parkinson's disease are characterized by unnatural motor movements. Most frequently these symptoms are manifested in the form of tremor, bradykinesia and/or rigidity of a subject's upper extremities. There is no cure yet for many movement disorders but an early and accurate detection is helpful. Such detection tools and techniques have been developed in the past but they have several drawbacks. In some tools and techniques, they involve imaging systems (e.g., CT, MRI, SPECT, or PET), electromyography systems, and/or neurological assessments by medical practitioners including tests for assessing cognitive functions. These tools and techniques are expensive, time-consuming, and troublesome and require the presence of a medical practitioner because movement disorder patients are generally unaware of his deficits and the medical reports and medical equipment need to be interpreted and operated by a doctor. Patients also need to travel to a medical institution where the scanning, measurements, and/or neurological assessments are conducted, which should be discouraged as they may already have a movement disorder and may further increase the chance of being injured.

In some tools and techniques, they involve using accelerometers and gyroscopes to quantify movement disorder symptoms. These sensors, however have limitations. Accelerometers operate in response to the local gravitational field, and therefore they often have problems in separating changes in linear acceleration from rotation. Further, results of a second integration required to obtain linear position are often contaminated with noise, making measurement difficult at best. Gyroscopes measure angular velocity independent of gravity with a good frequency response. However, static angular position cannot be measured accurately due to DC drift characteristic with these devices.

Therefore, there is a need for less expensive, convenient, fast, and effective diagnostic tools and measures which can be carried out in a simple manner during daily life situations by the affected patients and other individuals.

### SUMMARY OF THE INVENTION

In accordance with principles of the invention, a computer-implemented method for determining whether an individual has a movement disorder is contemplated. The method comprises a pattern receiving and converting software application to be implemented on a mobile device. After this application is implemented on the mobile device, it configures the mobile device to receive a user trace made on a pattern; establish a X-Y coordinate plane and convert the received trace into a plurality of coordinates using the X-Y coordinate plane; and transmit the plurality of coordinates representing the received trace to a computer system over a communications network. The method also comprises a pattern processing application to be implemented on a computer system. After this application is implemented on the computer system, it configures the computer system to receive the plurality of coordinates representing the received trace over the communications network; reconstruct a user trace image using the received plurality of coordinates; and implement a machine learning system configured to determine an index having a value for the reconstructed user trace that represents both whether the user has a movement disorder and the severity of his movement disorder if he does. The machine learning system is implemented by transforming images of traces produced by individuals into a format to be used by the machine learning system in configuring a neural network in the machine learning system; and configuring the neural network in the machine learning system to produce a category label using the transformed images, wherein the category label outputs the index having a value. The pattern processing application also configures the computer system to transmit the determined index having a value to the mobile device to inform the user whether he has a movement disorder and the severity of his movement disorder if he does.

In accordance with principles of the invention, a computer-implemented method for determining whether an individual has a movement disorder is contemplated. The method comprises implementing a movement disorder determination software application on one or more electronic devices. The one or more electronic devices include a microprocessor and memory configured to store computer instructions executable by the microprocessor. The software application includes computer instructions to be stored in the memory that are executable by the microprocessor to perform computer-implemented steps. The steps comprise receiving a user trace made on a pattern; establishing a X-Y coordinate plane and converting the received trace into a plurality of coordinates using the X-Y coordinate plane; reconstructing a user trace image using the received plurality of coordinates; and implementing a machine learning system configured to determine an index having a value for the reconstructed user trace that represents both whether the user has a movement disorder and the severity of his movement disorder if he does. The machine learning system is implemented by transforming images of traces produced by individuals into a format to be used by the machine learning system in configuring a neural network in the machine learning system; and configuring the neural network in the machine learning system to produce a category label using the transformed images, wherein the category label outputs the index having a value. A non-transitory computer readable medium storing the above movement disorder determination software application that causes a computer to execute the above method is also contemplated.

In one embodiment, the value of the index has a first range between two numbers indicating the likelihood a movement disorder is present in the reconstructed user trace image. One of the two numbers represents that a movement disorder is not present in the reconstructed user trace image. Another one of the two numbers represents that a movement disorder is present in the reconstructed user trace image. The value of the index has a second range between another two numbers indicating severity of a movement disorder.

In one embodiment, the step of transforming images of traces produced by individuals includes images of traces produced by individuals without a movement disorder and images of traces produced by individuals with a movement disorder. The step of transforming images of traces produced by individuals includes images of traces produced by individuals with Parkinson's disease.

In one embodiment, the step of transforming includes transforming the images of traces produced by individuals into a two-tone format that turns the images being transformed into two-tone images, employing a first threshold color value to turn image points in the images being transformed above the first threshold color value into a first color, and employing a second threshold color value to turn image points in the images being transformed below the second threshold color value into a second color.

In one embodiment, each of the image points has a corresponding pixel array value (x, y, z) and the pixel array value (x, y, z) is adjusted to (255, 255, 255) when the average of the color values of all the pixels in the array is above the first threshold color value.

In one embodiment, each of the image points has a corresponding pixel array value (x, y, z) and the pixel array value (x, y, z) is adjusted to (0, 0, 0) when the average of the color values of all the pixels in the array is below the second threshold color value.

In one embodiment, the step of configuring a neural network in the machine learning system to produce a category label includes configuring in softmax layer in the neural network to produce a category label.

In one embodiment, the pattern includes a Archimedean spiral.

In accordance with principles of the invention, a non-transitory computer readable medium storing an application that causes a computer to execute a method is contemplated. The method comprises implementing a movement disorder determination software application including a pattern receiving and converting software application to be implemented on a mobile device and a pattern processing software application to be implemented on a computer system. The mobile device and the computer system include a microprocessor, memory storing computer instructions executable by the microprocessor, and a network interface operatively coupled to the microprocessor allowing the mobile device and the computer system to communicate with each other over a communications network.

The pattern receiving and converting software application includes computer instructions to be stored in the memory of the mobile device that are executable by the microprocessor of the mobile device to perform computer-implemented steps. The steps comprise receiving a user trace made on a pattern; establishing a X-Y coordinate plane and converting the received trace into a plurality of coordinates using the X-Y coordinate plane; and transmitting the plurality of coordinates representing the received trace to a computer system over the communications network.

The pattern processing software application includes computer instructions to be stored in the memory of the computer system that are executable by the microprocessor of the computer system to perform computer-implemented steps. The steps comprise receiving the plurality of coordinates representing the received trace over the communications network; reconstructing a user trace image using the received plurality of coordinates; and implementing a machine learning system configured to determine an index having a value for the reconstructed user trace that represents both whether the user has a movement disorder and the severity of his movement disorder if he does. The machine learning system is implemented by transforming images of traces produced by individuals into a format to be used by the machine learning system in configuring a neural network in the machine learning system; and configuring the neural network in the machine learning system to produce a category label using the transformed images, wherein the category label outputs the index having a value. The pattern processing software application includes computer instructions to be stored in the memory of the computer system that are executable by the microprocessor of the computer system to further perform computer-implemented steps comprising transmitting the determined index having a value to the mobile device to inform the user whether he has a movement disorder and the severity of his movement disorder if he does.

In one embodiment, the step of transforming includes transforming the images of traces produced by individuals into a two-tone format that turns the images being transformed into two-tone images, employing a first threshold color value to turn image points in the images being transformed above the first threshold color value into a first color, and employing a second threshold color value to turn image points in the images being transformed below the second threshold color value into a second color.

In one embodiment, each of the image points has a corresponding pixel array value (x, y, z) and the pixel array value (x, y, z) is adjusted to (255, 255, 255) when the average of the color values of all the pixels in the array is above the first threshold color value.

In one embodiment, each of the image points has a corresponding pixel array value (x, y, z) and the pixel array value (x, y, z) is adjusted to (0, 0, 0) when the average of the color values of all the pixels in the array is below the second threshold color value.

In accordance with principles of the invention, a movement disorder determination system is contemplated. The system comprises a microprocessor and memory storing computer instructions executable by the microprocessor, wherein the system is implemented with a movement disorder determination software application that includes computer instructions stored in the memory of the system that are executable by the microprocessor of the system to perform computer-implemented steps. The steps comprise receiving a user trace made on a pattern; establishing a X-Y coordinate plane and converting the received trace into a plurality of coordinates using the X-Y coordinate plane; reconstructing a user trace image using the plurality of coordinates; and implementing a machine learning system configured to determine an index having a value for the reconstructed user trace that represents both whether the user has a movement disorder and the severity of his movement disorder if he does. The machine learning system is implemented by transforming images of traces produced by individuals into a format to be used by the machine learning system in configuring a neural network in the machine learning system; and configuring the neural network in the machine learning system to produce a category label using the transformed images, wherein the category label outputs the index having a value. The movement disorder determination software application includes computer instructions stored in the memory of the system that are executable by the microprocessor of the system to further perform computer-implemented steps comprising transmitting the determined index having a value to the mobile device to inform the user whether he has a movement disorder and the severity of his movement disorder if he does.

In one embodiment, the step of transforming includes transforming the images of traces produced by individuals into a two-tone format that turns the images being transformed into two-tone images, employing a first threshold color value to turn image points in the images being transformed above the first threshold color value into a first color, and employing a second threshold color value to turn image points in the images being transformed below the second threshold color value into a second color In one embodiment, each of the image points has a corresponding pixel array value (x, y, z) and the pixel array value (x, y, z) is adjusted to (255, 255, 255) when the average of the color values of all the pixels in the array is above the first threshold color value.

In one embodiment, each of the image points has a corresponding pixel array value (x, y, z) and the pixel array value (x, y, z) is adjusted to (0, 0, 0) when the average of the color values of all the pixels in the array is below the second threshold color value.

### BRIEF DESCRIPTION OF THE DRAWINGS

The nature and various advantages of the present invention will become more apparent upon consideration of the following detailed description, taken in conjunction with the accompanying drawings, in which like reference characters refer to like parts throughout, and in which:

FIG. 1 depicts an illustrative movement disorder determination system in accordance with some embodiments of the present invention;

FIG. 2 depicts an illustrative a user interface configured to present a pattern and receiving a user trace in accordance with some embodiments of the present invention;

FIG. 3 depicts an illustrative X-Y coordinate plane configured to convert a user's trace into coordinates in accordance with some embodiments of the present invention;

FIG. 4 depicts an illustrative machine learning system in accordance with some embodiments of the present invention; and

FIGS. 5a-5b depict an illustrative transformation process in accordance with some embodiments of the present invention.

### DETAILED DESCRIPTION OF THE INVENTION

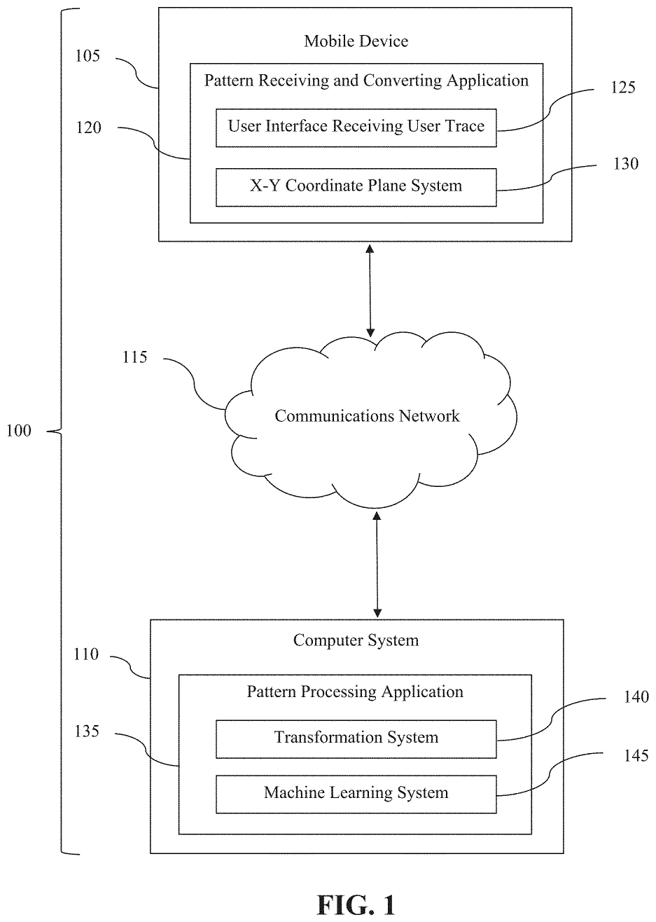

FIG. 1 depicts an illustrative movement disorder determination system 100. The system 100 comprises a mobile device 105 implemented with a pattern receiving and conversion software application 120 and a computer system 110 implemented with a pattern processing software application 135. The mobile device 105 and the computer system 100 are configured to transmit and receive data over a communications network 115. The pattern receiving and conversion software application 120 and the pattern processing software application 135 may be collectively referred to as a movement disorder determination software application. A user can trace a pattern provided by the pattern receiving and conversion software application 120 on the mobile device 105 and the computer system 110 implemented with the pattern processing software application 135 can determine whether the user has a movement disorder from the received trace and show the result to the user via the mobile device 105. The mobile device 105 can be other types of electronic devices having a microprocessor and memory such as a desktop computer, a laptop computer, a tablet computer, or a personal digital assistant (PDA). The computer system 110 can be a server.

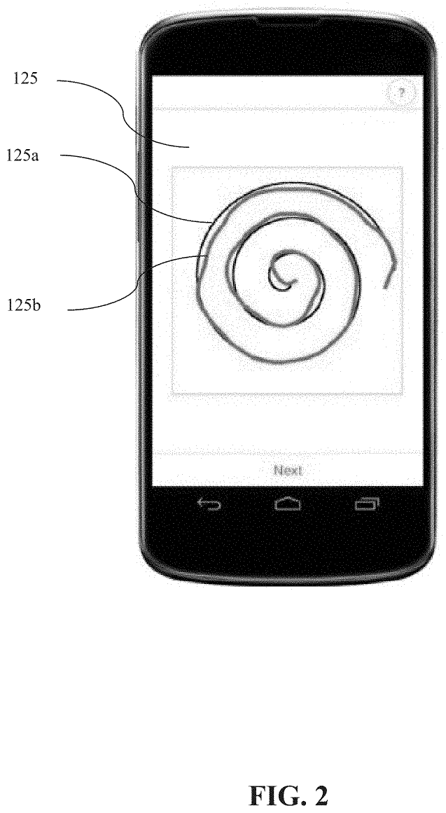

The pattern receiving and conversion software application 120 provides a user interface 125 allowing users to trace a pattern presented by the pattern receiving and conversion software application (or input a trace on the screen or pattern). Preferably, the pattern is a spiral or Archimedean spiral, but other patterns or shapes are also contemplated such as in the form of a character, number, or symbol. FIG. 2 depicts an illustrative user interface 125 showing a pattern (Archimedean spiral) 125a displayed by the application 120 and the user's trace 125b. The application 120 presents a pattern on the screen of the mobile device and the user can trace the pattern on the screen using his finger or a stylus, or by contacting the screen in other manners. The screen is preferably a touch-screen display but it can be other types of screens. The application 120 stores a plurality of patterns (e.g., pattern images having a pattern).

As the user traces the pattern, the application 120 may display or otherwise indicate the user's course of movement (e.g., display the pattern in one color and the user's trace or course of movement in another color). The areas on the screen contacted by the user are displayed (e.g., 125b). In some embodiments, the user's course of movement can be displayed after he finishes tracing, rather than in real-time. User's trace and user's course of movement are synonymous and are used interchangeably in this application.

The pattern receiving and conversion software application 120 can also receive the user's trace in other ways. For example, the application can receive an image with the user's trace on a pattern. Such an image may be obtained by photographing (e.g., using a camera on the mobile device to take a picture) or scanning a sheet having a pre-printed figure with the user's trace on it.

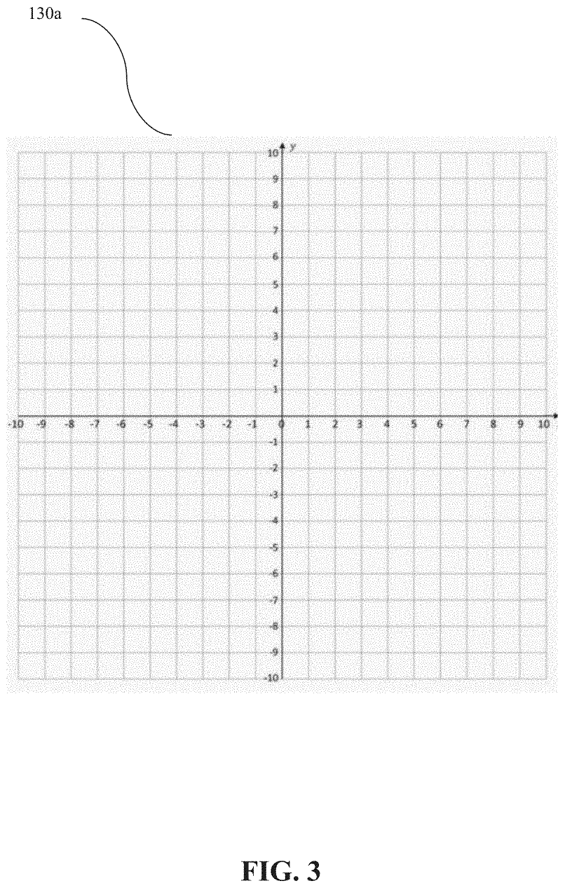

The pattern receiving and conversion software application 120 provides a x-y coordinate plane that is configured to record or convert the user's trace. The x-y coordinate plane is provided by a x-y coordinate plane system 130 of the application 120 and FIG. 3 depicts an illustrative x-y coordinate plane 130a. The x-y coordinate plane 130a defines a plurality of coordinates (e.g., (x₁, y₁), (x₂, y₁), (x₃, y₁) . . . (x₁, y₁), (x₁, y₂), (x₁, y₃) . . . (x₂, y₂) . . . (xₙ, yₙ)), and each coordinate (e.g., x₁, y₁) may correspond to a pixel (e.g., a red pixel) or a pixel array (e.g., a red pixel, a green pixel, and a blue pixel, or RGB array) in the screen. The x-y coordinate plane 130 may be incorporated into the pattern provided by the application 120, or be used by the application 120 to map the user's trace on the pattern to the x-y coordinate plane 130a. As such, the user's trace on the pattern can be recorded by the application 120 in the form of coordinates (or converted into coordinates). The user's trace can be converted into coordinates as the user traces the pattern or after the user finishes tracing the pattern. Pixels or pixel arrays contacted by the user or covered by the user's trace are detected by the application 120 and their corresponding coordinates are recorded. The resolution of the user's trace can be reduced first and then the application can determine the appropriate coordinates using the lower resolution user trace. This way, the application 120 can generate and transmit a smaller amount of coordinates without losing the pattern of the user's trace.

The coordinates are then transmitted to the computer system 110 implemented with a pattern processing software application 135 over the communications network 115. The recorded coordinates may be the only information representing the user's trace that is sent to the computer system 100. Transmission of an image or other visualization of the user's trace is not necessary. The computer system 110 may also communicate with the mobile device 105 over the communications network 115 such as transmitting the result of whether the user has a movement disorder (e.g., the index or indexes discussed below). The communication between the mobile device 105 and the computer system 110 can be implemented using a platform-based service or Platform as a Service (PaaS) such as Heroku, Azure, OpenShift, CloudFoundry, Amazon Web Service (e.g., Elastic Bean-Stalk), Engine Yard, Jelastic, or other services. In one embodiment, the communication is implemented using Heroku. The mobile device 105 (or the computer system 110) can make HTTP requests to the computer system 110 (or the mobile device 105) with the coordinates (or movement disorder determination result) as a payload.

From the received coordinates, the pattern processing software application 135 may construct the user's trace or an image of the user' trace. The application 135 implements a machine learning system 145 and the reconstructed user trace is fed to the machine learning system 145. The machine learning system 145 is configured to output a single index indicating both the likelihood a movement disorder is present in the reconstructed user trace (e.g., a value between 0 and 1, where 0 means the user has no movement disorder and 1 means the user has a movement disorder) and the severity of the movement disorder if a movement disorder is present in the reconstructed user trace (e.g., a value of 1.5, where 1 indicates that the user has a movement disorder and 0.5 indicates the severity of the movement disorder). In other words, and in one embodiment, the single index includes a first range indicating the likelihood a movement disorder is present in the reconstructed user trace and a second range indicating the severity of the movement disorder if a movement disorder is present in the reconstructed user trace. The value of the single index may further have other levels of measurements or increase such as double or triple to further indicate the individual's movement disorder severity. In some embodiments, two or more separate indexes can be used. The application 135 may also use the coordinates to determine the value of the single index without reconstructing the user's trace first (e.g., the coordinates, instead of the reconstructed user trace, are fed to the machine learning system). Using a combination of coordinates and images to determine the value of the single index is also contemplated. The value of the single index is transmitted to the mobile device 105 and displayed to the user showing him whether he has a movement disorder and the severity of his movement disorder if he does. The value of the single index is the movement disorder determination result, which includes the likelihood a movement disorder is present in the received user trace and a second range indicating the severity of the movement disorder if a movement disorder is the received user trace.

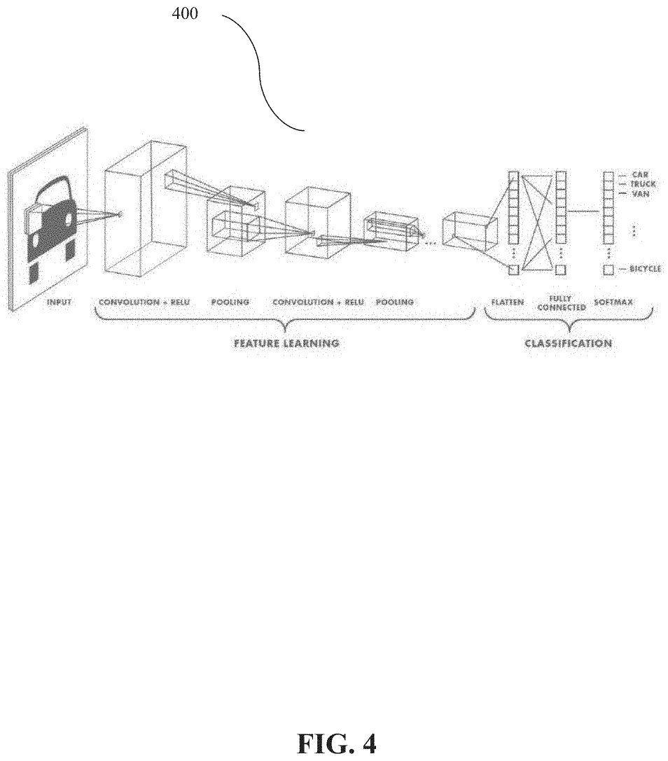

FIG. 4 depicts an illustrative machine learning system 400 includes a first artificial neural network (feature learning component) and a second artificial neural network (classification component). The first neural network is configured to receive a user trace or an image of a user trace, detect features in the image (e.g., edges, shapes, colors, and/or other characteristics of the trace in the image), and compute a vector representing the detected features. The first neural network is implemented using a convolution layer, a rectified linear unit (ReLu) layer, and a pooling layer. The first neural network may include multiple convolution layers, ReLu layers, and pooling layers to compute the features vector. The convolution layer, ReLu layer, and pooling layer perform multiple iterations (e.g., on a scale of thousands or hundreds of thousands) to determine the final features vector.

The output of the first neural network is connected to the input of the second neural network (classification component). The second neural network includes category labels implemented on a softmax layer and is configured to classify the features vector to one of the labels using a fully connected layer. The second neural network classifies the features vector to the appropriate category label by determining the likelihood or percentage that the features vector falls into each category label (e.g., a classification score) and assigning the features vector to the category label with the highest percentage (e.g., 1% of chance that the features vector falls into category label car, 6% of chance that the features vector falls into category label truck, and 93% of chance that the features vector falls into category label van, and assigning the features vector to category label van). In one embodiment of the invention, only one category label is implemented on the softmax layer and the second neural network is configured to classify the features vector to only that label using the fully connected layer. That category label is the one that indicates the likelihood a movement disorder is present in the user' trace and the severity of the movement disorder if a movement disorder is present in the user' trace (or the movement disorder determination result).

By classifying or assigning the features vector to the one category label, the training image for which the features vector is computed is also classified to that category label. The index or value of that category label can indicate the likelihood of having a movement disorder and the severity of the movement disorder if a movement disorder is present in the training image.

The first neural network and the second neural network can include recurrent neural networks (e.g., long short-term memory recurrent neural networks), feed-forward neural networks, convolutional neural networks (e.g., MobileNets), deep neural networks, multi-layer non-linear models, or other forms of neural networks. The first neural network and the second neural network can also be implemented in other manners such as logistic regression and support vector machines (SVM).

The machine learning system 145 is trained by using images of traces produced by individuals over a pattern. In one embodiment, the images include images of traces produced by individuals over a pattern without a movement disorder and images of traces produced by individuals over a pattern with a movement disorder. In the latter set of images, it includes traces produced by individuals having different movement disorder severity. Both set of images are supplied to the machine learning system 145 from the first neural network or the convolution layer in the first neural network. The machine learning system is trained until can distinguish the traces produced by individuals with and without a movement disorder to a very high degree of accuracy (e.g., above 90%). The training allows the machine learning system 145 to determine an index value for a received user trace, which can be received in the form of an image or coordinates. In other words, the images of traces produced by individuals over a pattern are used to configure a neural network in the machine learning system to produce a category label. The category label is configured to output an index value.

Movement disorder may be Parkinson's disease or other diseases such as multiple sclerosis, Alzheimer's disease, Hunting's disease, ataxia, dystonia, myasthenia, and epilepsy, or a disorder caused by such diseases. When the machine learning system is implemented to determine whether a user has a movement disorder caused by Parkinson's disease, the machine learning system is trained by using images of traces produced by individuals over a pattern without movement disorder and images of traces produced by individuals over the pattern with movement disorder caused by Parkinson's disease. The pattern being traced is preferably a spiral or Archimedean spiral. The machine learning system can be trained by using the appropriate images or traces and patterns depending on the type of movement disorder or the disease underlying the movement disorder the machine learning system seeks to determine.

Each of the images used to train the machine learning system, whether they are produced by individuals without movement disorder or individuals with movement disorder, is transformed into a format to be used by the machine learning system in configuring a neural network in the machine learning system. In one embodiment, the images to be transformed are transformed into a two-tone format or two-tone images (e.g., each is a black and white image) by a transformation system 140 before images are supplied to the machine learning system 145. An image of trace may refer to an image produced by scanning or photographing a sheet with a pre-printed pattern and a trace on the pattern (e.g., a piece of paper with a pre-printed pattern and a trace on the pattern drawn by pen or pencil). Before transformation, the training images may be color images. In some embodiments, the images to be transformed may be transformed into multiple-tone images (three color images, four color images, or more).

In one embodiment of the transformation process 140, the pattern processing software application 135 analyzes each image and determines the color of each point in the image (e.g., which can be the smallest unit or the smallest set of units that can be perceived by the screen of the mobile or the pattern processing software application). The color of each point can be determined by a color value of a pixel array (e.g., a RGB array which comprises or consists of a red pixel, a green pixel, and a blue pixel). The color value of a pixel array has a range between and including (0, 0, 0), which is color black, and (255, 255, 255), which is color white. Each pixel array is configured to produce light, and the color of light is determined by a color value controlled by the screen or the pattern receiving and conversion software application. By determining the color value that is needed to drive or light up a pixel array, the color of the corresponding point can also be determined.

After the color value of each pixel array in the image is determined, every determined color value that has a color value above a first threshold is adjusted to (255, 255, 255), and every determined color value that has a color value below a second threshold is adjusted to (0, 0, 0). The first and second threshold color values are two different values with one used to turn a color value or pixel array to white and another one used to turn a color value or pixel array to black. The transformation process can remove other all other color than the specified two colors from the image. In one embodiment, the first threshold is color value 180 and the second threshold is color value 130. Each threshold can be based on a color value of any pixel in the array, the average color value of the three pixels in the array, or require the color value of each pixel in the array to meet the threshold. For example, when the average color value is used, a pixel array having a color value of (192, 0, 125) is set to (0, 0, 0) because its average color value is 106, and a pixel array having a color value of (192, 160, 225) is set to (255, 255, 255) because its average color value is 192. For another example, if the user's trace was drawn using a blue pen, then the user's trace is turned into black after the transformation process. If the pattern was pre-printed on a piece of paper in a color other than black such as blue, then the pattern is turned into black after the transformation process. If the user's trace and/or the pattern were already in black, then they would stay in black or still be turned into black. It is understood that the pixel array may comprise or consist of other color and number of pixels such as CMYK and the above concepts equally apply.

Additional thresholds may be employed depending on how many tones or colors the transformed images should have. The number of thresholds may correspond to the number of tones or colors. Other transformation processes are also contemplated.

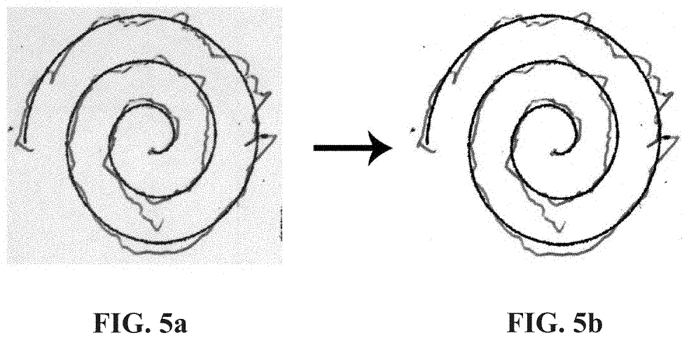

FIGS. 5a-b depict an illustrative transformation process. FIG. 5a depicts an image of trace (which can be drawn by either a healthy individual or a diseased individual) before transformation. FIG. 5b depicts the image after transformation. The image in FIG. 5a may be a color image. The image in FIG. 5b is a two-tone image (e.g., a black and white image) with colors other than the specified two tones or colors removed from the image.

The transformed images may be used to configure a neural network in the machine learning system to produce a category label. The category label is configured to output an index value.

The resolution of the images used to train the machine learning system can be adjusted such as lowered before or after the transformation process. The resolution of the images used to train the machine learning system and the resolution of the reconstructed user trace images are preferably the same or similar (if not the same, one or both of them are adjusted to the same or similar resolution). Same or similar resolution may allow the pattern processing software application to more easily determine movement disorder and severity.

From testing, it has been found that a machine learning system trained with the transformed images provides high accuracy (e.g., above 90%) in determining whether a user have a movement disorder such as a movement disorder caused by Parkinson's disease and/or severity. For example, using a set of images of traces produced by healthy individuals, the machine learning system found more than 90% of those individuals do not have a movement disorder. For another example, using a set of images of traces produced by diseased individuals, the machine learning system found more than 90% of those individuals have a movement disorder. For yet another example, using a set of images of traces produced by healthy individuals and diseased individuals, the machine learning system has an accuracy of more than 90% in determining whether the individual of the provided image has a movement disorder.

In some embodiments, the pattern processing software application may also use the aforementioned x-y coordinate plane to convert the images of traces from healthy individuals and diseased individuals into coordinates representing the corresponding image and machine learning system can be trained using those coordinates as opposed to images. Using a combination of coordinates and images to train the machine learning system is also contemplated.

The pattern processing software application is an application that processes the received user pattern to determine whether the individual produced the pattern has a movement disorder (or the likelihood of the individual having a movement disorder) and the severity of that individual's movement disorder if he does. The term process or its equivalents mean providing the received user pattern (whether in the form of a reconstructed image or coordinates) to the machine learning machine and the machine learning machine determining the value of the single index for the received user pattern.

In some embodiments, the pattern receiving and conversion software application and the pattern processing software application can be implemented on the same electronic device such as on the mobile device 105. Therefore, receiving the user trace and processing the received user trace can be done locally without communicating with another computer system such as a server or access point such as a router.

The pattern receiving and conversion software application and the pattern processing software application can be implemented using a computer programming language including but not limited to, Javascript, Java, Objective-C, C, C++, C#, PHP, Python, Swift, and other computer programming languages. In one embodiment, the pattern receiving and conversion software application is implemented using Swift whereas the pattern processing software application is implemented using Python.

Communications network may be a network that uses a suitable communications protocol such as Wi-Fi, 802.11, Bluetooth, radio frequency systems such as 900 MHz, 1.4 GHz, and 5.6 GHz communication systems, infrared, GSM, GSM plus EDGE, CDMA, quadband, and other cellular protocols, VOIP, or any other suitable protocol. The network may be established wirelessly or by using wires such as an optical fiber or Ethernet cable.

Each of the mobile device, computer system, server, and other devices (each may be referred to as an electronic device or a computer) described in this application may include a microprocessor, memory, storage media, display (or screen), and network interface.

The microprocessor may be an application specific integrated circuit (ASIC), programmable logic array (PLA), digital signal processor (DSP), field programmable gate array (FPGA), or any other integrated circuit. The microprocessor may also be other types of processors, such as a system-on-a-chip that combines one or more of a CPU, an application processor, and memory, or a reduced instruction set computing (RISC) processor.

The memory is coupled to the microprocessor for temporarily storing information and instructions to be executed by the microprocessor. For example, the memory may comprise a random access memory (RAM) or cache memory.

The storage media refers to a non-transitory media that store information and/or instructions to be executed by the microprocessor. Such storage media may be non-volatile media, for example, comprising a floppy disk, a flexible disk, hard disk, solid state drive, magnetic tape, or any other magnetic data storage medium, a CD-ROM, any other optical data storage medium, any physical medium with patterns of holes, a PROM, and EPROM, or a FLASH-EPROM.

The display (or screen) may comprise a cathode ray tube (CRT), touch screen, or other monitors for displaying, receiving, and/or entering information.

The network interface may be a hardware device configured to support the aforementioned communications protocols such as integrated services digital network (ISDN) card, local area network (LAN) card, Ethernet card, cable modem, satellite modem, or a modem to provide a data communication connection to a corresponding type of telephone line.

Server means a communication-oriented computer; usually with fast internal clock, large memory, large storage capacity, and general capability of sustaining concurrent data communication with multiple end users or client devices. Server may comprise, by way of example, network computers or other types of computers or processing elements capable of being configured for the maintenance, storage, delivery or other processing of information received or deliverable over the Internet or the communications network.

The term system can refer to hardware system, software system, or a combination of hardware system and software system.

It is understood that discussion with respect to software applications also apply to the underlying mobile device and computer system on which the software application is implemented, and vice versa.

Counterpart method and computer-readable medium embodiments would be understood from the above and the overall disclosure.

It is understood from the above description that the functionality and features of the systems, devices, or methods of embodiments of the present invention include generating and sending signals to accomplish the actions.

Exemplary systems, devices, and methods are described for illustrative purposes. Further, since numerous modifications and changes will readily be apparent to those having ordinary skill in the art, it is not desired to limit the invention to the exact constructions as demonstrated in this disclosure. Accordingly, all suitable modifications and equivalents may be resorted to falling within the scope of the invention. Broader, narrower, or different combinations of the described features are also contemplated, such that, for example features can be removed or added in a broadening or narrowing way. It should be understood that combinations of described features or steps are contemplated even if they are not described directly together or not in the same context. Applications of the technology to other fields are also contemplated.

Thus, for example, any sequence(s) and/or temporal order of steps of various processes or methods (or sequence of device connections or operation) that are described herein are illustrative and should not be interpreted as being restrictive. Accordingly, it should be understood that although steps of various processes or methods or connections or sequence of operations may be shown and described as being in a sequence or temporal order, but they are not necessarily limited to being carried out in any particular sequence or order. For example, the steps in such processes or methods generally may be carried out in various different sequences and orders, while still falling within the scope of the present invention. Moreover, in some discussions, it would be evident to those of ordinary skill in the art that a subsequent action, process, or feature is in response to an earlier action, process, or feature.

It is also implicit and understood that the applications or systems illustratively described herein provide computer-implemented functionality that automatically performs a process or process steps unless the description explicitly describes user intervention or manual operation.

The terms or words that are used herein are directed to those of ordinary skill in the art in this field of technology and the meaning of those terms or words will be understood from terminology used in that field or can be reasonably interpreted based on the plain English meaning of the words in conjunction with knowledge in this field of technology. This includes an understanding of implicit features that for example may involve multiple possibilities, but to a person of ordinary skill in the art a reasonable or primary understanding or meaning is understood.

Software application can be implemented as distinct modules or can be integrated together into an overall application such as one that includes the user interface and that handles other feature for providing the functionality to the user on their device.

The words “may” and “can” are used in the present description to indicate that this is one embodiment but the description should not be understood to be the only embodiment.

It should be understood that claims that include fewer limitations, broader claims, such as claims without requiring a certain feature or process step in the appended claim or in the specification, clarifications to the claim elements, different combinations, and alternative implementations based on the specification, or different uses, are also contemplated by the embodiments of the present invention

It is intended that the specification and examples be considered as exemplary only, with a true scope being indicated by the claims and their equivalents.

What is claimed is:

1. A computer-implemented method for determining whether an individual has a movement disorder comprising:
implementing a movement disorder determination software application on one or more electronic devices, the one or more electronic devices include a microprocessor and memory configured to store computer instructions executable by the microprocessor, and the software application includes computer instructions to be stored in the memory that are executable by the microprocessor to perform computer-implemented steps comprising:
receiving a user trace made on a pattern;
establishing a X-Y coordinate plane and converting the received trace into a plurality of coordinates using the X-Y coordinate plane;
reconstructing a user trace image using the received plurality of coordinates; and
implementing a machine learning system configured to determine an index having a value for the reconstructed user trace that represents both whether the user has a movement disorder and the severity of his movement disorder if he does;
wherein the machine learning system is implemented by:
transforming images of traces produced by individuals into a format to be used by the machine learning system in configuring a neural network in the machine learning system; and
configuring the neural network in the machine learning system to produce a category label using the transformed images, wherein the category label outputs the index having a value.

2. The method of claim 1, wherein the value of the index has a first range between two numbers indicating the likelihood a movement disorder is present in the reconstructed user trace image.

3. The method of claim 2, wherein one of the two numbers represents that a movement disorder is not present in the reconstructed user trace image.

4. The method of claim 2, wherein another one of the two numbers represents that a movement disorder is present in the reconstructed user trace image.

5. The method of claim 2, wherein the value of the index has a second range between another two numbers indicating severity of a movement disorder.

6. The method of claim 1, wherein the step of transforming images of traces produced by individuals includes images of traces produced by individuals without a movement disorder and images of traces produced by individuals with a movement disorder.

7. The method of claim 6, wherein the step of transforming images of traces produced by individuals includes images of traces produced by individuals with Parkinson's disease.

8. The method of claim 1, wherein the step of transforming includes transforming the images of traces produced by individuals into a two-tone format that turns the images being transformed into two-tone images, employing a first threshold color value to turn image points in the images being transformed above the first threshold color value into a first color, and employing a second threshold color value to turn image points in the images being transformed below the second threshold color value into a second color.

9. The method of claim 8, wherein each of the image points has a corresponding pixel array value (x, y, z) and the pixel array value (x, y, z) is adjusted to (255, 255, 255) when the average of the color values of all the pixels in the array is above the first threshold color value.

10. The method of claim 8, wherein each of the image points has a corresponding pixel array value (x, y, z) and the pixel array value (x, y, z) is adjusted to (0, 0, 0) when the average of the color values of all the pixels in the array is below the second threshold color value.

11. The method of claim 1, wherein the step of configuring a neural network in the machine learning system to produce a category label includes configuring in softmax layer in the neural network to produce a category label.

12. The method of claim 1, wherein the pattern includes a Archimedean spiral.

13. A non-transitory computer readable medium storing an application that causes a computer to execute a method, the method comprising:
implementing a movement disorder determination software application on one or more electronic devices, the one or more electronic devices include a microprocessor and memory configured to store computer instructions executable by the microprocessor, and the software application includes computer instructions to be stored in the memory that are executable by the microprocessor to perform computer-implemented steps comprising:
receiving a user trace made on a pattern;
establishing a X-Y coordinate plane and converting the received trace into a plurality of coordinates using the X-Y coordinate plane;
reconstructing a user trace image using the received plurality of coordinates; and
implementing a machine learning system configured to determine an index having a value for the reconstructed user trace that represents both whether the user has a movement disorder and the severity of his movement disorder if he does;
wherein the machine learning system is implemented by:
transforming images of traces produced by individuals into a format to be used by the machine learning system in configuring a neural network in the machine learning system; and
configuring the neural network in the machine learning system to produce a category label using the transformed images, wherein the category label outputs the index having a value.

14. The method of claim 13, wherein the step of transforming includes transforming the images of traces produced by individuals into a two-tone format that turns the images being transformed into two-tone images, employing a first threshold color value to turn image points in the images being transformed above the first threshold color value into a first color, and employing a second threshold color value to turn image points in the images being transformed below the second threshold color value into a second color.

15. The method of claim 14, wherein each of the image points has a corresponding pixel array value (x, y, z) and the pixel array value (x, y, z) is adjusted to (255, 255, 255) when the average of the color values of all the pixels in the array is above the first threshold color value.

16. The method of claim 14, wherein each of the image points has a corresponding pixel array value (x, y, z) and the pixel array value (x, y, z) is adjusted to (0, 0, 0) when the average of the color values of all the pixels in the array is below the second threshold color value.

17. A movement disorder determination system comprising:
a microprocessor and memory configured to store computer instructions executable by the microprocessor, wherein the system is implemented with a movement disorder determination software application that includes computer instructions stored in the memory that are executable by the microprocessor to perform computer-implemented steps comprising:
receiving a user trace made on a pattern;
establishing a X-Y coordinate plane and converting the received trace into a plurality of coordinates using the X-Y coordinate plane;
reconstructing a user trace image using the plurality of coordinates; and
implementing a machine learning system configured to determine an index having a value for the reconstructed user trace that represents both whether the user has a movement disorder and the severity of his movement disorder if he does;
wherein the machine learning system is implemented by:
transforming images of traces produced by individuals into a format to be used by the machine learning system in configuring a neural network in the machine learning system; and
configuring the neural network in the machine learning system to produce a category label using the transformed images, wherein the category label outputs the index having a value.

18. The system of claim 17, wherein the step of transforming includes transforming the images of traces produced by individuals into a two-tone format that turns the images being transformed into two-tone images, employing a first threshold color value to turn image points in the images being transformed above the first threshold color value into a first color, and employing a second threshold color value to turn image points in the images being transformed below the second threshold color value into a second color

19. The system of claim 18, wherein each of the image points has a corresponding pixel array value (x, y, z) and the pixel array value (x, y, z) is adjusted to (255, 255, 255) when the average of the color values of all the pixels in the array is above the first threshold color value.

20. The system of claim 18, wherein each of the image points has a corresponding pixel array value (x, y, z) and the pixel array value (x, y, z) is adjusted to (0, 0, 0) when the average of the color values of all the pixels in the array is below the second threshold color value.
